(19) United States
(12) Patent Application Publication
(10) Pub. No.: US 2006/0041266 A1
(43) Pub. Date: Feb. 23, 2006

(54) Surgical scalpel

(76) Inventors: Stephen J. Sullivan, (US); Myles Kobren, (US); Andrew Menzin, (US)

Correspondence Address: ELIOT GERBER, 127 DEVIN DR., MORAGA, CA, 94556, US

(21) Appl. No.: 11/193,568

(22) Filed: Aug. 1, 2005

Related U.S. Application Data
(63) Continuation-in-part of application No. 29/211,438, filed on Aug. 17, 2004.

(51) Int. Cl. A61B 17/32

(52) U.S. Cl. 606/167

(57) ABSTRACT

A surgical scalpel has a flat blade, which may have a cutting or severing edge, and an elongated ergonomic handle having distal and proximal ends and an imaginary central axis. In one preferred embodiment, of a disposable (one use) scalpel the blade is embedded in a molded plastic resin handle. The handle has a generally triangle shape in cross-section with the top face being slightly curved. The handle, near its distal end, has an enlarged finger pressure section having an indentation at its top face adapted for a finger pad to apply pressure to the scalpel handle.

### CROSS REFERENCE TO RELATED APPLICATIONS

The present application is a continuation-in-part application partly based upon design application 29/211438 filed Aug. 17, 2004, which is titled “Surgical Scalpel” and which was filed in the names of Stephen Sullivan, Myles Kobren and Andrew Menzin.

### FIELD OF THE INVENTION

The present invention relates to medical devices and more particularly to surgical scalpels.

### BACKGROUND OF THE INVENTION

At the present time, medical instruments known as surgical scalpels are used as a knife by physicians and surgeons in many medical situations. In office use, they are used for many purposes such as skin incisions. They are commonly used in surgical procedures to sever skin and other body portions. They are available with various lengths of handles and shapes and sizes of blades. Generally, disposable scalpels comprise a flat handle (proximal portion) and a non-detachable sharp blade (distal portion) and sometimes a retractable shield.

Scalpels are often used only once “disposable” and their handles may be plastic. Other scalpels use a scalpel handle which removably attaches to a disposable surgical blade and the handle, for example of stainless steel, may be sterilized for re-use.

This general type of flat-handled scalpel has been in common use for many years without any noticeable improvements to the commercially available handles. However the United States patent literature shows many prior attempts to improve surgical scalpels. For example, various prior patents are directed to the issue of blade safety, to prevent the blade from cutting the surgeon or nurse. Generally, these patents suggest that the blade be retracted into the handle and then projected when it is to be used or that a protective shield by positioned over the blade and the shield withdrawn prior to its use. In U.S. Patent Application 2003/009395 to Dambal et al the blade is retractable within a hollow generally flat handle. In U.S. Pat. No. 5,312,429 to Noack a blade release slide element is used to replace blades. In U.S. Patent Application 2002/0143352 to Newman et al a shield is slid on the handle to expose the blade. In U.S. Pat. No. 6,500,187 to Petersen the handle is in two parts that are coupled in alternative configurations. In U.S. Patent Application 2004/0098001 to Holman the blade is retractable to within the handle.

In addition, the patent literature shows various shapes for scalpel handles. In U.S. Des. 329,699 to Schutte et al two blades are fixed to a fork-like scalpel handle. In U.S. Pat. No. 5,571,127 to De Camli a generally flat handle is said to be ergonomic. In U.S. Patent application 2004,0097999 to Wilson a scalpel handle, said to be “anatomic”, has two opposite curved portions In U.S. Des. 327,125 the top and sides, at the distal end of the handle, has ridges.

### BRIEF SUMMARY OF THE INVENTION

The present invention is of a surgical scalpel and more specifically the invention is directed to the shape of an ergonomic handle for a scalpel. “Ergonomic” means how an instrument can be best designed for comfort, safety, efficiency and productivity and is also called “human factor engineering”. In the case of a scalpel, the ergonomic handle of the present invention permits a physician/surgeon to position the blade more exactly in terms of its angle and its direction; to apply a more exactly determined pressure and to perform incisions and other procedures with reduced hand and body fatigue. In one disposable embodiment of the scalpel of the present invention, a metal knife scalpel blade is embedded in the distal end (away from the hand) of a plastic resin handle. The handle is elongated and has an imaginary longitudinal axis. Generally cross-sections of the handle (taken perpendicular to the axis) are triangular. The scalpel handle has generally a top parallel to the dull edge of the blade, and two generally flat inclined side faces. The handle has an indented slight concave at its top, at the handles enlarged finger pressure section, for finger pressure.

### BRIEF DESCRIPTION OF THE SEVERAL VIEWS OF THE DRAWING

The following detailed description of the invention should be taken in conjunction with the accompanying drawing.

In the drawing:

FIG. 1 is a perspective view of a disposable scalpel which is an embodiment of the invention;

FIG. 2 is a right side view of the scalpel of FIG. 1;

FIG. 3 is a left side view of the scalpel of FIG. 1;

FIG. 4 is a top view of the scalpel of FIG. 1;

FIG. 5 is a bottom view of the scalpel of FIG. 1;

FIG. 6 is a front view of the scalpel of FIG. 1;

FIG. 7 is a rear view of the scalpel of FIG. 1;

FIG. 8 is a cross-sectional view of the scalpel of FIG. 1 taken along line A-A of FIG. 3 looking in the direction of the arrows.

### DETAILED DESCRIPTION OF THE INVENTION

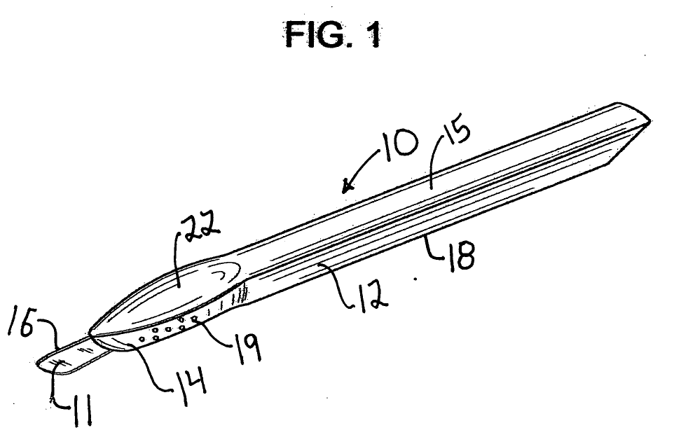

The present invention is of a surgical scalpel and the preferred embodiment is the disposable (one-use) scalpel 10 shown in FIGS. 1-8.

A scalpel blade 11 is fixed at the distal end of scalpel 10. In the embodiment shown in FIGS. 1-8 the blade 11 is embedded in the handle 12. However, alternatively in non-disposable embodiments (not shown) the blade 11 may be removable and replaceable, using conventional blade fastening means (not shown). The blade 11 may be of a suitable size and shape for the function of the scalpel. For example the blade may be of a conventional size and shape such as a carbon or stainless steel blade numbers 10, 11, 12, 13, 14, 15, 18, 20, 21, 22, 23, 24 or 25. Alternatively the blade may be the special blade for, for example for caesarean sections (“C-Sections”) as in U.S. Pat. No. 6,102,924 having a blunt or rounded edge to divide or separate tissue. This is the type of blade illustrated as blade 11 in FIGS. 1-8. The blade 11 has a sharper severing edge 23 and a duller bottom non-severing top edge 16.

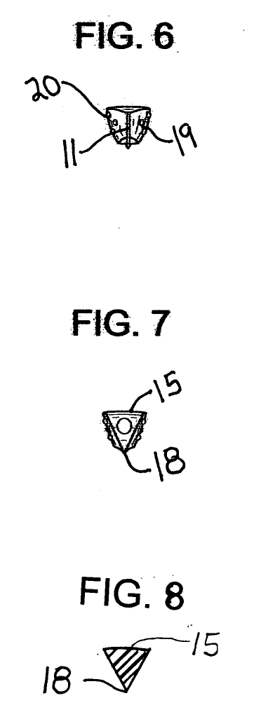

The handle 12 is elongated and has an imaginary central axis 13, shown in FIG. 8, which runs its length. The handle, in cross-section as shown in FIG. 8, and as indicated in FIGS. 6 and 7, except for its expanded distal portion 14, is generally a triangle. The top face 15 is slightly curved and is parallel with the top edge 16 (the non-cutting edge) of blade 11. The right side face 12 and the left side face 17 form a “V” (acute angle in the range of 20-60 degrees) having a tip line 18.

The distal section 14, is slightly enlarged and called herein the “finger pressure section”, has small protrusions (bumps) at its right 19 and left 20 side walls. The finger pressure section is about one-third the length of the entire handle. The finger pressure section 14 has a slight bottom curvature 21 and an indentation 22 at its top face. That indentation 22 is about as wide and long as the pad of an adult's index finger, e.g. the pad from the tip of the index finger to the first knuckle.

Preferably the handle 12 is molded, as a single solid unitary piece, by injection molding and is preferably a suitable plastic resin such as high impact polypropylene, ABS or high impact polyethylene. The handle 12 may be colored with different colors to indicate different sizes and/or shapes of blades.

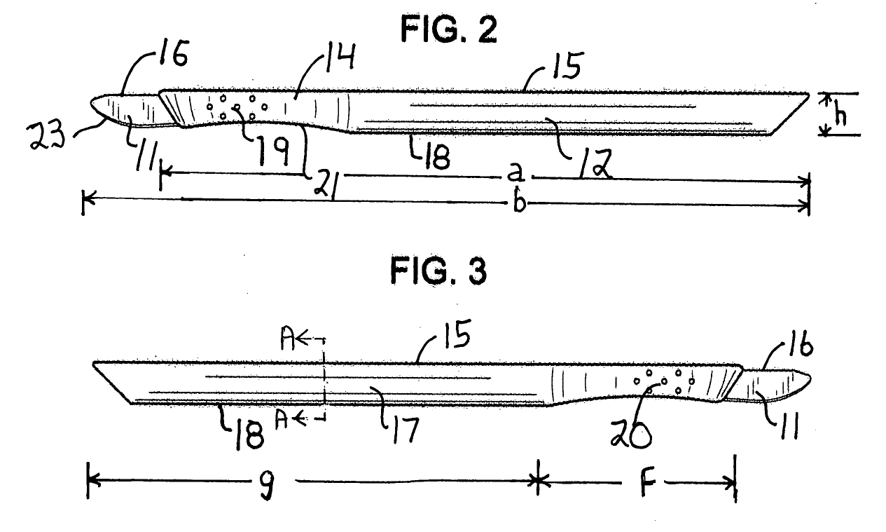

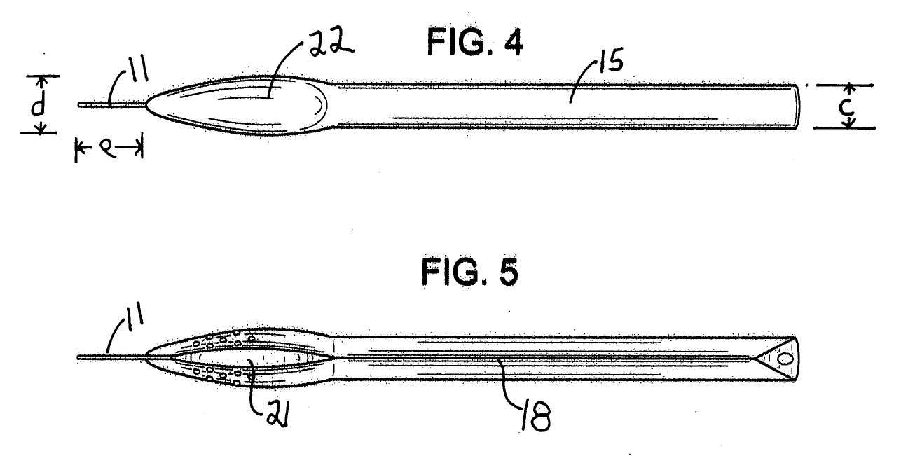

In the preferred embodiment, as exemplified by a prototype, the dimensions are as follows: included acute angle between sides 12-17 is 30 degrees; length of entire scalpel (b in FIG. 2) is 6.72 inches (170.75 mm); length of handle (a in FIG. 2) is 6.10 inches (155 mm); width of top face 15 at proximal end (body section) (c in FIG. 4) is 0.37 inches (9.43 mm); width of enlarged portion near distal end of handle as seen from above as in FIG. 4 (finger pressure section) (d in FIG. 4) is 0.52 inches (13 mm); length of scalpel blade (e in FIG. 4) is 0.62 inches (15.75 mm); length of finger pressure section (f in FIG. 3) is 1.81 inches (46 mm); length of body section (g in FIG. 3) is 4.29 inches (109 mm); and height (h in FIG. 2) is 0.44 inches (11.24 mm). The blade is a stainless steel. #10 “C-section” blade as described in U.S. Pat. No. 6,102,924. The concavity 22 is 0.04 inches (1 mm) deep. There are 7 left side bumps 19 and 7 right side bumps 20k. Each bump is a hemisphere of 0.04 inches (1 mm) diameter.

All of the above-mentioned patent documents are incorporated by reference herein.

What is claimed is:

1. A surgical scalpel comprising a blade and a handle, the blade having a severing edge and top non-severing edge, the blade and handle being connected, characterized in that:
a. the handle is an elongated ergonomic unitary member having an imaginary central axis and distal and proximal ends, a top face and two side faces angled with respect to the top face;
b. the handle has an elongated body section and a finger pressure section which is unitary therewith and which is proximate the handle's distal end, the body section being triangular, in cross-section taken perpendicular to the axis; and
c. the finger pressure section has a top face and a concavity in the top face adapted to receive the front pad of an average index finger;

2. A surgical scalpel as in claim 1 where the severing edge of the blade is dull and specifically adapted for a C-Section surgical operation.

3. A surgical scalpel as in claim 1 wherein the handle is an injection molded plastic resin and the blade is embedded in the handle.

4. A surgical scalpel as in claim 1 wherein the triangular cross-sections of the body portion is a triangle having an outward curvature at the top face.

5. A surgical scalpel as in claim 1 wherein the finger pressure section is about one-third of the length of the handle and the body section is about two-thirds of said length.

6. A surgical scalpel as in claim 1 wherein the handle has a top face which is parallel with the blade's non-severing edge.

7. A surgical scalpel as in claim 6 wherein the top face of the finger pressure section is wider than the top face of the body portion.

8. A surgical scalpel comprising a blade and a handle, the blade having a severing edge and a top non-severing edge, the blade and handle being connected, characterized in that:
a. the handle is an elongated ergonomic unitary member having an imaginary central axis and distal and proximal ends, a top face and two side faces angled with respect to the top face;
b. the handle has an elongated body section and a finger pressure section, the body section is proximate the handle's proximal end and the finger pressure section is proximate the handle's distal end; and
c. the finger pressure section has a concavity in the top face adapted to receive the front pad of an average index finger.

9. A surgical scalpel as in claim 8 wherein the severing edge of the blade is dull and specifically adapted for a C-Section surgical operation.

10. A surgical scalpel as in claim 8 wherein the handle is an injection molded plastic resin and the blade is embedded in the handle.

11. A surgical scalpel as in claim 8 wherein the body portion has a top face and cross-sections of the body portion taken perpendicular to the axis are generaly trianglar and having a curvature at the top face.

12. A surgical scalpel as in claim 8 wherein the finger pressure section is about one-third of the length of the handle and the body section is about two-thirds of said length.

13. A surgical scalpel as in claim 11 wherein the top face of the handle is parallel with the blade's non-severing edge.

14. A surgical scalpel as in claim 11 wherein the finger pressure section has a top face which is wider than the top face of the body portion.

15. A surgical scalpel comprising a blade, the blade having a severing edge and top non-severing edge, and a handle, the blade and handle being connected, characterized in that:
a. the handle is an elongated ergonomic unitary member having an imaginary central axis and distal and proximal ends, a top face, and two side faces angled with respect to the top face;
b. the handle, proximate its distal end, has a finger pressure section with a top face, the top face having an indentation which is adapted to receive the front pas of an average index finger; and
c. the handle has an elongated body section unitary with the finger pressure section and proximate the handle's proximal end, the body section being triangular in cross-sections taken perpendicular to the axis.
